(19) United States
(12) Patent Application Publication
(10) Pub. No.: US 2024/0371483 A1
(43) Pub. Date: Nov. 7, 2024

(54) SYSTEM AND METHOD FOR AUTOMATICALLY GENERATING REPORT FOR ULTRASOUND IMAGING EXAMINATION

(71) Applicant: KONINKLIJKE PHILIPS N.V., EINDHOVEN (NL)

(72) Inventor: Earl M. Canfield, II, New Braunfels, TX (US)

(21) Appl. No.: 18/648,741

(22) Filed: Apr. 29, 2024

Related U.S. Application Data
(60) Provisional application No. 63/463,709, filed on May 3, 2023.

(51) Int. Cl. G16H 15/00; G06F 3/0484; G06F 40/166; G06F 40/40

(52) U.S. Cl. CPC G16H 15/00; G06F 3/0484; G06F 40/166; G06F 40/40

(57) ABSTRACT

A system for generating a report from ultrasound imaging includes an ultrasound imaging system configured to provide ultrasound image data from examination of a subject, and a server configured to execute a trained, AI driven large language model. The ultrasound imaging system includes a processor configured to receive the ultrasound image data, generate an ultrasound image, and generate a data set indicative of measurements in the ultrasound image. Also included is a display and a GUI including a report button selectable by a user to request a report. A prompt string generator is configured to receive the user input, extract a type of examination and the data set from the processor, translate them into a natural language prompt string, and output the prompt string to an API readable by the AI driven large language model. The AI driven large language model automatically generates the report for display.

### BACKGROUND

Artificial intelligence (AI) large language models have been developed, examples of which include ChatGPT available from OpenAI and Bard available from Google. Generally, AI large language models are designed to understand natural language queries and to generate natural language text response based on these queries, making them capable of participating in conversations, answering questions, and assisting with various tasks. The AI large language models leverage a vast knowledge base accumulated over multiple years, enabling them to provide relevant and contextual information across numerous domains. By employing sophisticated machine learning techniques, including deep learning and natural language processing, the AI large language models have the ability to interact with users intelligently, adapt to diverse contexts, and even exhibit creative problem-solving.

However, AI large language models are underutilized in the field of ultrasound imaging, such as understanding, interpreting, and diagnosing ultrasound imaging data and aiding physician workflow. This is largely due to the challenge of providing relevant ultrasound imaging and patient data in a format that consistently results in cogent and successful AI large language model outputs.

### SUMMARY

According to a representative embodiment, a system is provided for generating a report from ultrasound imaging. The system includes an ultrasound imaging system configured to provide ultrasound image data from an examination of a subject, and a server configured to execute one or more trained, artificial intelligence (AI) driven large language models (LLM) configured to generate natural language responses to natural language input. The ultrasound imaging system includes at least one processor configured to receive the ultrasound image data acquired by an ultrasound imaging device during the examination of the subject, including corresponding metadata, to generate an ultrasound image based on the ultrasound image data, and to generate a data set indicative of multiple measurements in the ultrasound image; a display configured to display the ultrasound image and the data set; a graphical user interface (GUI) provided on the display, the GUI including a report selector selectable by a user to request a report regarding the examination of the subject and one or more configurable elements for receiving user input from the user; and a prompt string generator configured to receive the user input from the GUI, to extract at least a type of examination and the data set from the at least one processor, to translate the user input, the type of examination, and the data set into a natural language prompt string in response to selection of the report selector, and to output the prompt string to an application programming interface (API), where the prompt string is suitable for the type of examination and readable by the AI driven large language model. The AI driven large language model receives the prompt string via the API and automatically generates the report suitable for the type of examination and the user input, where the report includes a summary of the data set. The display is further configured to receive and display the report from the AI driven large language model.

According to another representative embodiment, a non-transitory computer readable medium stores instructions for generating a report from ultrasound imaging that, when executed by one or more one processors, cause the one or more processors to receive ultrasound image data acquired by an ultrasound imaging device during an examination of a subject, the ultrasound image data including corresponding metadata; generate an ultrasound image based on the ultrasound image data; generate a data set indicative of multiple measurements in the ultrasound image; cause the ultrasound image and the data set to be displayed on a display; receive a request for a report regarding the examination of the subject from a GUI in response to selection by a user of a report selector displayed by the GUI; receive user input from the user in response to one or more configurable elements displayed by the GUI; translate the user input, the data set and a type of examination into a natural language prompt string in response to the selection of the report selector, where the prompt string is suitable for the type of examination and readable by a trained, AI driven large language model configured to generate natural language responses to the natural language prompt string; output the prompt string to an API configured to interface with the AI driven large language model; automatically generate the report suitable for the type of examination and the user input using the AI driven large language model, where the report includes a summary of the data set; and cause the report generated by the AI driven large language model to be displayed on the display.

According to another representative embodiment, a method is provided for generating a report from ultrasound imaging. The method includes receiving ultrasound image data acquired by an ultrasound imaging device during an examination of a subject, the ultrasound image data including corresponding metadata; generating an ultrasound image based on the ultrasound image data; generating a data set indicative of multiple measurements in the ultrasound image; displaying the ultrasound image and the data set on a display; receiving a request for a report regarding the examination of the subject from a GUI on the display in response to selection by a user of a report selector displayed by the GUI; receiving user input from the user in response to one or more configurable elements displayed by the GUI; translating the user input, the data set and a type of examination into a natural language prompt string in response to the selection of the report selector, where the prompt string is suitable for the type of examination and readable by a trained, AI driven large language model configured to generate natural language responses to the natural language prompt string; outputting the prompt string to an API configured to interface with the AI driven large language model; generating the report suitable for the type of examination and the user input using the AI driven large language model, where the report includes a summary of the data set; and displaying the report generated by the AI driven large language model on the display.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for generating a report from ultrasound imaging, according to a representative embodiment.

FIG. 2 shows an example of a GUI for providing information to populate a prompt string, according to a representative embodiment.

FIG. 3 is a simplified a block diagram of an ultrasound imaging system including a system for generating a report from ultrasound imaging, according to a representative embodiment.

FIG. 4 is a flow diagram of a method for generating a report from ultrasound imaging, according to a representative embodiment.

FIG. 5 is a simplified block diagram of a representative processing unit, according to a representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms “a,” “an” and “the” are intended to include both singular and plural forms, unless the context clearly dictates otherwise. Additionally, the terms “comprises,” and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

As used in the specification and appended claims, and in addition to their ordinary meanings, the term “about” and “approximately” mean to with acceptable limits or degree. For example, “approximately 2 MHz” means one of ordinary skill in the art would consider the signal to be 2 MHz within reasonable measure. Also, as used in the specification and appended claims, in addition to its ordinary meaning, the term “substantially” means within acceptable limits or degree. For example, the term “substantially simultaneously” means one of ordinary skill in the art would consider occurrence at the same time.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

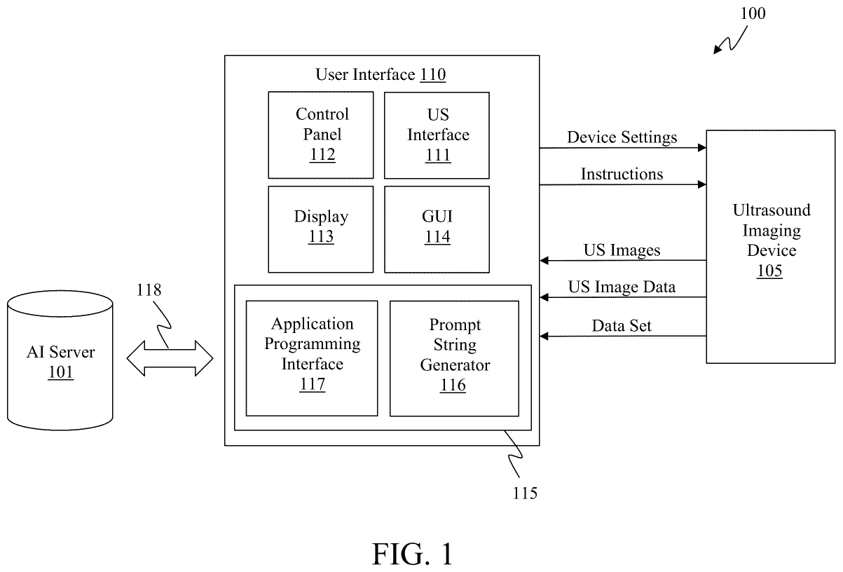

FIG. 1 is a simplified a block diagram of an ultrasound imaging system including a system for generating a report from ultrasound imaging, according to a representative embodiment.

Referring to FIG. 1, ultrasound imaging system 100 includes a user interface 110, an ultrasound imaging device 105, and an artificial intelligence (AI) server 101 accessible to the user interface 110 by one or more communication channels 118. The one or more communication channels 118 may be wired (e.g., Ethernet, USB) or wireless (e.g., Bluetooth, Wi-Fi) communication channels, for example. Also, the one or more communication channels 118 may be implemented over a wide area network (e.g., internet, cloud) or a local area network (e.g., Ethernet).

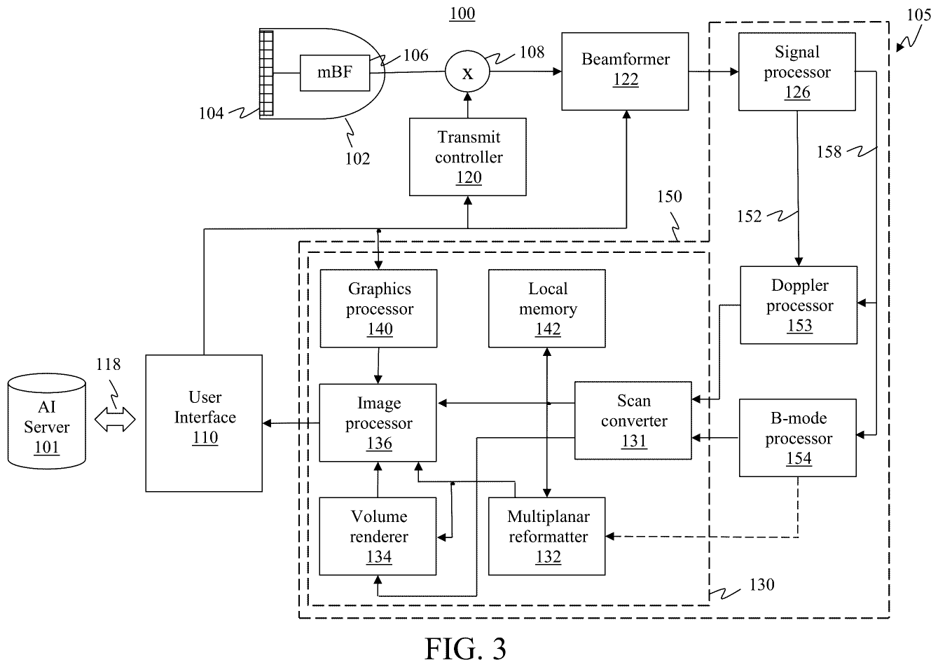

The ultrasound imaging device 105 is configured to acquire ultrasound images from examination of a subject (e.g., patient) under control of the user interface 110, which interfaces with the ultrasound imaging device 105 via known US interface 111. The ultrasound imaging device 105 receives basic device settings from the user interface 110, such as gain, time gain compensation (TCG), focal zone, frequency, gray scale, and dynamic range, as well as exam specific data, such as type of examination, user name, and patient name, for example. Further, during the examination, the ultrasound imaging device 105 may receive user instructions, such as caliper settings indicating locations of desired measurements in the ultrasound images and/or annotations labeling or highlighting the measurements and/or specified features in the ultrasound images. The ultrasound imaging device 105 generates a data set corresponding to the ultrasound images indicating responses to the user instructions. For example, the data set may include the results of the desired measurements, and may associate the annotations with specified locations in the ultrasound images. The ultrasound imaging device 105 is described in more detail below with reference to FIG. 3.

The user interface 110 further includes a control panel 112, a display 113, and a graphical user interface (GUI) 114. The control panel 112 enables the user to enter data and to control or manipulate aspects of the processes described herein, as well as control or manipulation of the ultrasound imaging device 105. Although depicted separately, it is understood that all or a portion of the control panel 112 may be implemented by the GUI 114, which is displayed on the display 113. The control panel 112 includes one or more mechanical controls, touch sensitive controls, and/or other known input devices. For example, the mechanical controls may include one or more of a mouse, a keyboard, a trackball, a joystick, a microphone, a video camera, buttons, encoders, voice or gesture recognition captured by a microphone and video camera, and the touch sensitive controls may include one or more of a touchpad, a trackpad, and a touchscreen (e.g., on the display 113). The control panel 112 enables the user to provide the device settings and instructions to the ultrasound imaging device via the US interface 111.

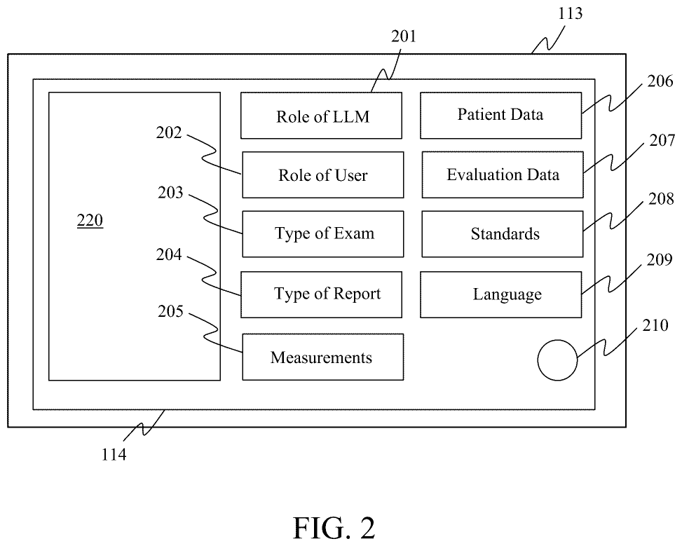

The display 113 may be a monitor such as a computer monitor, a television, a liquid crystal display (LCD), an organic light emitting diode (OLED), a flat panel display, a solid-state display, or a cathode ray tube (CRT) display, or an electronic whiteboard, for example. The display 113 includes a screen for viewing the ultrasound images and associated data sets, as well as the GUI 114 to enable the user to interact with the displayed images and other interactive features. The display 113 enables display of all or part the information provided to the user interface 110 via the US interface 111, including the ultrasound images, the ultrasound image data associated with the ultrasound mages (including metadata), and the data sets responsive to the user's instructions. The metadata in particular includes context information, such as identification of the ultrasound imaging device 107, the settings, exam times, and the like. The GUI 114 provides multiple visual elements enabling the user to interact with the user interface 110, including a report selector selectable by the user to request a report regarding the examination of the subject, such as a diagnostic report, and one or more configurable elements for receiving user input from the user. For example, the report selector may be a button, a check box, a toggle, or other visual indicia of requesting the report. An example of the GUI 114 is shown in FIG. 2, discussed below.

The user interface 110 also includes prompt string generator 116 and application programming interface (API) 117. The prompt string generator 116 and the API 117 may be implemented by processing unit 115, an example of which is discussed below with reference to FIG. 8. Alternatively, one or both of the prompt string generator 116 and the API 117 may be implemented by one or more processors provided in the ultrasound imaging device 105, examples of which are discussed below with reference to FIG. 3.

The prompt string generator 116 is configured to translate data received from the ultrasound imaging device 105 and the GUI 114 into a preformatted template for a prompt string or query that is compatible with a trained AI driven large language model (e.g., machine learning algorithm) implemented by the AI server 101. The prompt string is a text string that includes roles and other context information in a preamble, and includes a data set in a body of the prompt string. The context information may include one or more of the roles of the LLM and the user, the type of examination being reported, applicable industry standards and language. The data set includes measurements, for example, and may be part of the prompt string or may be in a data file included with or referenced by the prompt string. The prompt string is provided to the API 117, e.g., as a text file, which interfaces with the AI server 101. In response, the AI server executes the trained AI large language model to generate a formatted textual response returned to the user interface 110, as discussed below. In an embodiment, one or more images may also be provided to the API 117 as a separate image file.

More particularly, the prompt string generator 116 is configured to receive user input from the GUI 114, to extract at least the type of examination and the data set provided by the ultrasound imaging device 105, and to translate the user input, the type of examination, and the data set into a natural language text string in a predetermined template. The type of examination and the data set may be extracted by the prompt string generator 116 from a natural language input or from predetermined configurable fields displayed on the GUI 114, which prompt the specific information desired for the predetermined template, discussed below. The prompt string generator 116 outputs the prompt string to the API 117, which in turn interfaces with the AI server 101 over the one or more communication channels 118. The prompt string is suitable for the type of examination and is readable by the AI driven large language model implemented by the AI server 101. The prompt string generator 116 may initiate generation of the prompt string in response to user input entered at the GUI 114, such as selection of the report button, for example.

FIG. 2 shows an example of a GUI for providing information to populate a prompt string, according to a representative embodiment. Referring to FIG. 2, the display 113 displays the GUI 114 on a screen (e.g., touch screen) to be viewed by the user. In the depicted configuration, GUI 114 includes ultrasound image 220 from the ultrasound imaging device 105, enabling the user to interact directly with the ultrasound image 220. The GUI 114 further includes report button 210 (selector) that is selectable by the user to initiate generation of the prompt string by the prompt string generator 116. Alternatively, the GUI 114 may automatically indicate to the prompt string generator 116 to initiate generation of the prompt string in response to the user indicating completion of the ultrasound imaging examination, in which case the report button 210 is optional.

The GUI 114 also includes illustrative configurable fields that are completed by the user for use by the prompt string generator 116 in generating the prompt string. In the depicted example, the configurable fields are Role of LLM field 201, Role of User field 202, Type of Exam field 203, Type of Report field 204, Measurements field 205, Patient Data field 206, Evaluation Data field 207, Standards field 208, and Language field 209.

The Role of LLM field 201 is the role assumed by the trained AI driven large language model in analyzing the prompt string, such as “ultrasound specialist,” “fetal ultrasound specialist,” or “ultrasound technician,” for example, to direct the focus and depth of the analysis. The Role of the User field 202 is the profession and/or position of the person entering the information and possibly performing and/or analyzing the ultrasound examination, such as “physician” or “sonographer,” for example, to direct the focus and depth of the response output by the AI driven large language model. The Role of the User field 202 may include additional information to add further context, such as years of experience, training, and/or specialty of the user. Each of the Role of LLM field 201 and the Role of User field 202 may be a text field for the user to enter the information or a selection field with available choices, such as a drop down list, for example.

The Type of Exam field 203 identifies the type of examination being performed that produces the ultrasound image data, such as an “obstetric ultrasound exam,” “high risk obstetric ultrasound exam” or a “prenatal ultrasound exam,” for example. This informs the AI driven large language model as to the focus and priorities of the imaging. The Type of Report field 204 indicates the format and detailed content of the response to be output by the AI driven large language model, such as “final report,” “intra-examination report,” or “over-read checklist,” for example. The Type of Report field 204 may also enable selection of a person other than the user to be the report recipient, such as the patient themselves, which would affect the content of the report. Each of the Type of Exam field 203 and the Type of Report field 204 may be a text field for the user to enter the information or a selection field with available choices, such as a drop down list, for example. The contents of the Role of the LLM field 201, the Role of the User field 202, the Type of Exam field 203, and the Type of Report field 204 may be included in the preamble of the prompt text, for example.

The Measurements field 205 enables the user to enter measurements made manually and/or automatically during the examination. Alternatively, or in addition, the user may simply select the Measurements field 205 element, which causes the GUI 114 and/or the prompt string generator 116 to retrieve the measurements directly from the ultrasound machine or a previously populated database of measurements, for example. In an obstetric ultrasound examination, for example, the measurements may include standard fetal measurements, such as biparietal diameter (BPD), occipital-frontal diameter (OFD), head circumference (HC), transcranial Doppler (TCD), transverse abdominal diameter (TAD), anterior-posterior abdominal diameter (APAD), abdominal circumference (AC) 18.96 cm, and femur length (FL), for example. Of course, other measurements may be included without departing from the scope of the present teachings. The contents of the Measurements field 205 may be included in the data set, which is included in the body of the prompt text, for example.

The Patient Data field 206 enables the user to enter specific information regarding the subject of the ultrasound imaging examination, such as age, weight, gender, vital signs, and/or all or part of the subject's medical history. Alternatively, or in addition, the user may simply select the Patient Data field 206 element, which causes the GUI 114 and/or the prompt string generator 116 to retrieve patient data directly from a patient database, such as an electronic health record (EHR) database, for example. The patient information may be anonymized before inclusion in the prompt string to protect the subject's privacy. Alternatively, the prompt string generator 116 may be configured to automatically retrieve all or a predetermined portion of the patient data when triggered to generate the prompt string. The Evaluation Data field 207 enables the user to indicate additional information about the examination. For example, when the type of examination is an obstetric ultrasound exam, the Evaluation Data field 207 indicates background information regarding the fetus, such as the number of fetuses (1, 2, 3, etc.), presentation (cephalic, breech), fetal heart rate and whether cardiac activity has been observed, for example. Alternatively, or in addition, the user may simply select the Evaluation Data field 207 element, which causes the GUI 114 and/or the prompt string generator 116 to retrieve the evaluation data directly from a patient database, such as an EHR database, for example. The patient information and/or the additional information about the examination may be included in the body of the prompt string generated by the prompt string generator 116.

The Standards field 208 indicates any standards relevant to the type of examination and the type of report. For fetal ultrasound in particular, the Standards field 208 may indicate the Hadlock age estimation table and the Hadlock weight estimation table as being applicable. In an embodiment, the standards may be fixed for each report, in which case there is no need for the Standards field 208. The Language field 209 indicates the language in which the report is to be written, such as “English,” “Spanish” or “German,” for example. The Language field 209 may be a separate field, as shown, or may be included as part of another field, such as the Type of Report field 204. One or more of the above configurable fields may have corresponding default settings.

Although the illustrative GUI 114 shows a selectable button and four text fields, it is understood that any type of configurable elements may be used, such as drop-down menus, check boxes, toggles, and radio buttons, for example, to acquire desired information without departing from the scope of the present teachings. Also, alternative or additional configurable elements may be included, such as providing a logo, title or other identifying information, for example, to be included in the response. In an alternative embodiment, the GUI 114 may provide a single configurable field, e.g., in addition to the report button 210, configured to receive a natural language text input from the user identifying desired features of the report, without departing from the scope of the present teachings.

The prompt string generator 116 may be configured to receive the entries from the GUI 114, or from corresponding databases, as indicated above, and to translate them into a predetermined format that satisfies input requirements of the AI driven large language model in order to produce the desired output. As discussed above, the text string may include a preamble that provides contextual information, such as the role to be assumed by the AI driven large language model, the role of the user, the type of examination and the type of report desired.

An example of a text string generated by the prompt string generator 116 further to a current patient exam may be as follows: “You are a fetal ultrasound specialist. I am an ultrasound sonographer. I am performing an ultrasound exam on a patient with a LMP of Mar. 1, 2023. These are the measurements completed. BPD 5.94 cm, OFD 7.57 cm, HC 21.33 cm, TCD 2.69 cm, TAD 5.82 cm, APAD 6.35 cm, AC 18.96 cm, FL 3.86 cm. Use the OB Hadlock growth table to assess these measurements. Create an OB exam report from this data, including the estimated due date and fetal weight.”

In this example, the role of the AI driven large language model in assessing the data (fetal ultrasound specialist), the role of the user (ultrasound sonographer), and patient specific data (last menstrual period (LMP) date of Mar. 1, 2023) may be received from the GUI 114, for example. The type of examination (fetal ultrasound) and the actual measurements (BPD 5.94 cm, OFD 7.57 cm, HC 21.33 cm, TCD 2.69 cm, TAD 5.82 cm, APAD 6.35 cm, AC 18.96 cm, and FL 3.86 cm) are in the data set retrieved from the data set provided by ultrasound imaging device 105. The type of standard to employ (Hadlock growth table) is retrieved from device settings, which may be provided to the prompt string generator 116 by the GUI 114, as discussed above, or the data set provided by the ultrasound imaging device 105. The information in the text string may be formatted as an informal or draft report on the patient exam.

In an embodiment, the prompt string generated by the prompt string generator 116 may be displayed on the display 113 and assessed for accuracy by the user prior to providing the prompt string to the API 117. In this case, the user is able to revise the prompt string, as needed, using the GUI 114. The prompt string as revised is then provided to the API 117.

The measurements in the above example (e.g., BPD 5.94 cm, OFD 7.57 cm, HC 21.33 cm, TCD 2.69 cm, TAD 5.82 cm, APAD 6.35 cm, AC18.96 cm, FL 3.86 cm) may be made interactively by the user by setting measurement calipers during the ultrasound imaging examination using a transducer probe, such as ultrasound probe 102, discussed below. Alternatively, the ultrasound imaging system 100 may further include a currently known or later developed AI auto-measurement module (not shown) configured to apply each image frame of a live ultrasound exam to a machine learning auto-measurement model to automatically identify and measure predetermined parameters, including fetal BPD, OFD, HC, TCD, TAD, APAD, AC, and FL, when the correct anatomy is present in the image frames. When the user is satisfied with the results, the user displays the automated measurements and selects (e.g., presses) the report button for the prompt string generator 116 to auto-generate the prompt string accordingly.

The trained AI driven large language model implemented by the AI server 101 inputs the prompt string received via the one or more communication channels 118 and automatically outputs a natural language textual response that provides an assessment of the examination and a diagnostic analysis of the subject. The response may be in the form of a formal diagnostic report or similar report, for example, suitable for the type of examination and the user input. The report may include a summary of the data set, as well as information generated by the AI driven large language model, such as diagnostics based at least in part on the data set. The prompt string generator 116 translating the user input, the type of examination, and the data set into the natural language text string, together with interaction of the GUI 114, improves functionality of the AI driven large language model with respect to generating this report(s) for the ultrasound imaging. The natural language text string is formatted in such a way as to ensure that the AI driven large language model provides a comprehensive, germane report geared to the user's level of expertise and need. More generally, the embodiments herein improve the technical field of ultrasound examinations and automated reporting.

The response from the AI driven large language model is received by the user interface 110 via the API 117 and is displayed on the display 113. In some embodiments, the response may be assessed for accuracy by the user, and edited if necessary using the GUI 114. The response may be used for diagnostic purposes and as a guideline for completing the patient examination, for example. The format of the response (e.g., a diagnostic report, an intra-examination report, etc.) is a function of the training of the AI driven large language model, as discussed below. Although, in an embodiment, the user may specify the format of the response in the prompt string provided by the prompt string generator 116. Generally, the report is suitable for the type of examination and the user input, and includes at least a summary of the data set.

The AI server 101 implementing the trained AI driven large language model may be a remote server accessible over a wide area network, such as the internet or the cloud. Further, the AI driven large language model may be a publicly available large language model, such as ChatGPT available from OpenAI or Bard available from Google, for example. When the AI driven large language model is one of the publicly available large language models, the prompt string generator 116 may automatically include website, license and key information, for example, in a preamble of the prompt string to enable access to the AI driven large language model without additional input by the user. Alternatively, the trained AI driven large language model may be a customized large language model provided by a hospital, group of hospitals, or other organization(s) trained specifically for ultrasound imaging. In this case, the AI server 101 may be available over a local areal network, such as an Ethernet connection, or a wide area network.

An example of a diagnostic report output by the AI server 101 in response to the example text string generated by the prompt string generator 116, discussed above, and displayed on the display 113, is as follows:
Based on the data provided, the estimated gestational age using the Hadlock table is 28 weeks and 3 days, which is consistent with the reported gestational age. The other measurements reported in the fetal ultrasound report are as follows:
BPD (biparietal diameter)=5.79 cm
OFD (occipital-frontal diameter)=7.57 cm
HC (head circumference)=21.33 cm
TCD (transcranial Doppler)=2.69 cm
TAD (transverse abdominal diameter)=5.82 cm
APAD (anterior-posterior abdominal diameter)=6.35 cm
AC (abdominal circumference)=26.33 cm
FL (femur length)=5.57 cm
These measurements are used to assess fetal growth and development and to monitor for any potential complications. In this case, the measurements appear to be within the expected range for a fetus of 28 weeks and 3 days gestational age.
As a medical doctor, it is important to review the full ultrasound report and consider any other relevant medical information in order to provide appropriate care for the patient and the developing fetus.

The AI driven large language model may include and/or implement any one or more machine learning models, deep learning models, artificial intelligence algorithms, and/or neural networks (collectively, models) which analyze the natural language prompt strings from the prompt string generator 116 based on training data associating terms in the prompt strings with known or ground truth terms. In some examples, the AI driven large language model may include an LSTM, a DNN, a CNN, an RNN, an autoencoder neural network, an ANN, a U-net model, or the like, to identify the terms in the prompt strings and determine content and layout of the responses (e.g., diagnostic reports). The AI driven large language model may be implemented in hardware (e.g., neurons are represented by physical components) and/or software (e.g., neurons and pathways implemented in a software application) components.

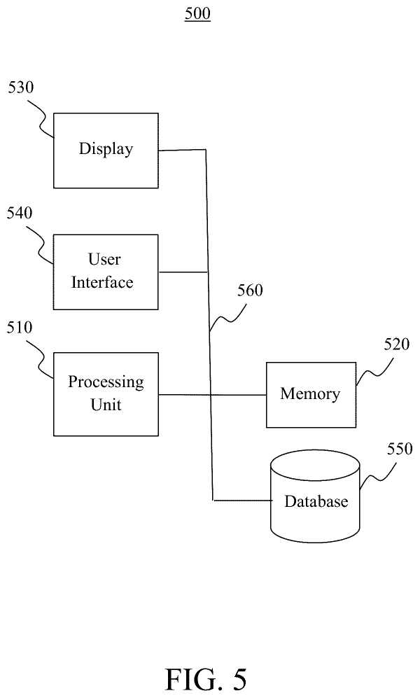

The AI driven large language model implemented according to the present disclosure may use a variety of topologies and learning algorithms for training the AI driven large language model to produce desired outputs. For example, a software-based neural network may be implemented by the AI server 101 using a processor (e.g., single or multi core CPU, a single GPU or GPU cluster, or multiple processors arranged for parallel-processing), as discussed below with reference to processor 510 in FIG. 5, configured to execute instructions, which may be stored in computer readable medium, and which when executed cause the processor to perform a trained algorithm for generating reports from the ultrasound imaging data sets.

In various embodiments, the AI driven large language model may be trained using any of a variety of currently known or later developed learning techniques to obtain a model (e.g., a trained algorithm, transfer function, or hardware-based system of nodes) that is configured to analyze prompt strings. Training data includes ultrasound imaging reports corresponding to ultrasound images and associated data, such as meta data, data sets, types of examinations, measurements and associated annotations, for example. In some embodiments, the AI driven large language model may be statically trained. That is, the AI driven large language model may be trained with a data set and deployed on the AI driven large language model. In some embodiments, the AI driven large language model may be dynamically trained. In these embodiments, the AI driven large language model may be trained with an initial data set and deployed on the ultrasound imaging system 100. However, the AI driven large language model may continue to train and be modified based on inputs acquired by the AI driven large language model after deployment of the AI driven large language model on the ultrasound imaging system 100.

When the AI driven large language model is implemented as a neural network, for example, the neural network may include several fully connected or convolutional layers with pooling, normalization, and non-linear layers between them, for example. When optional information about the patients (e.g., from EHRs) is included, this information may be incorporated by appending it to flattened feature layers before applying linear transformations followed by non-linearities, such as sigmoid functions and rectified linear units (ReLU), for example. The neural network may directly produce an nxl vector output, where n is the number of values regressed by the neural network. Errors are computed by comparing the output values produced by the neural network with ground truth values using some loss function, such as L1 or L2 loss, Huber loss, or log cosh loss, for example. The loss functions may be used to perform stochastic gradient descent to optimize network weights. This is typically done by computing derivatives of the loss function with respect to the model parameters using the chain rule. The derivatives inform the network how the model parameters must be adjusted through each training loop in order to minimize the loss function. The adjustments to model parameters are made starting from the last layer and working backwards in the network towards the first layer, which is known as backpropagation.

FIG. 3 is a simplified block diagram of an ultrasound imaging system including a system for generating a report from ultrasound imaging, according to a representative embodiment.

Referring to FIG. 3, in the depicted embodiment, ultrasound imaging system 100 includes an ultrasound probe 102, which may be an external probe or an internal probe such as an Intra Cardiac Echography (ICE) probe or a Trans Esophagus Echography (TEE) probe. The ultrasound probe 102 includes a transducer array 104 coupled to a microbeamformer 106. The transducer array 104 may be implemented as a flexible array configured to be conformably applied to a surface of subject to be imaged (e.g., patient). The transducer array 104 is configured to transmit ultrasound signals (e.g., beams, waves) into the subject and receive echo signals responsive to the transmitted ultrasound signals. Examples of the transducer array 104 include linear arrays, curved arrays, and phased arrays. The transducer array 104 may include a two dimensional array of transducer elements, for example, capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

The microbeamformer 106 is configured to control the transmission and reception of signals by the transducer elements in the transducer array 104. In some embodiments, the microbeamformer 106 may control the transmission and reception of signals by select active elements in the transducer array 104, such as a subset of active elements of the transducer array 104 that define an active aperture at any given time.

The ultrasound imaging system 100 further includes a transmit/receive (T/R) switch 108, a transmit controller 120, and a main beamformer 122. The microbeamformer 106 is coupled to the T/R switch 108, by a probe cable or wirelessly, for example. The T/R switch 108 is configured to switch between transmission for transmitting the ultrasound signals and reception for receiving the echo signals, and to protect the main beamformer 122 from high energy transmit signals. The T/R switch 108 may be included in the ultrasound probe 102 or in an ultrasound system base, which houses the image processing electronics in communication with the ultrasound probe 102. The ultrasound system base typically includes software and hardware components including circuitry for signal processing and image data generation, as well as executable instructions for providing a user interface (e.g., processing circuitry 150 and user interface 110).

Ultrasound signals from the transducer array 104 are transmitted under control of the microbeamformer 106, as directed by the transmit controller 120 coupled to the T/R switch 108 and the main beamformer 122. The transmit controller 120 is configured to control the direction in which beams are steered from the transducer array 104. For example, beams may be steered straight ahead from (orthogonal to) the transducer array 104, or at different angles for a wider field of view. The transmit controller 120 may also be coupled to the user interface 110 and receive input from the user's operation of the control panel 112, discussed above.

In some embodiments, partially beamformed signals produced by the microbeamformer 106 may be coupled to the main beamformer 122, which combines the partially beamformed signals from individual patches of transducer elements in the transducer array 104 into a fully beamformed signal. In some embodiments, the microbeamformer 106 is omitted, in which case the transducer array 104 is under the control of the main beamformer 122 which performs all beamforming of signals.

In embodiments with or without the microbeamformer 106, the beamformed signals of the main beamformer 122 are coupled to processing circuitry 150. The processing circuitry 150 includes a signal processor 126, a B-mode processor 154, and a Doppler processor 153, as well as image generation and processing components 130 configured to produce an ultrasound image from the beamformed signals (e.g., beamformed RF data). In the depicted embodiment, the image generation and processing components 130 include a scan converter 131, a multiplanar reformatter 132, a volume renderer 134, an image processor 136, a graphics processor 140, and a local memory 142.

The signal processor 126 is configured to process the received beamformed RF data in various ways, such as bandpass filtering, decimation, I and Q component separation, and harmonic signal separation. The signal processor 126 may also perform additional signal enhancement, such as speckle reduction, signal compounding, and noise elimination. The processed signals (also referred to as I and Q components or IQ signals) may be coupled to additional downstream signal processing circuits for image generation, discussed below. The IQ signals may be coupled to multiple signal paths, each of which may be associated with a specific arrangement of signal processing components suitable for generating different types of image data (e.g., B-mode image data, Doppler image data). For example, the ultrasound imaging system 100 may include a B-mode signal path 158 which couples the signals from the signal processor 126 to the B-mode processor 154 for producing B-mode image data.

The B-mode processor 154 may employ amplitude detection for imaging of structures in the subject. The signals produced by the B-mode processor 154 are coupled to the scan converter 131 and/or the multiplanar reformatter 132. The scan converter 131 is configured to arrange the echo signals from the spatial relationship in which they were received to a desired image format. For instance, the scan converter 131 may arrange the echo signal into a two dimensional (2D) sector-shaped format, or a pyramidal or otherwise shaped three dimensional (3D) format. The multiplanar reformatter 132 is configured to convert the echo signals received from points in a common plane in a volumetric region of the subject's body into an ultrasonic image (e.g., a B-mode image) of that plane, as described for example by Detmer in U.S. Pat. No. 6,443,896, which is hereby incorporated by reference in its entirety. The scan converter 131 and multiplanar reformatter 132 may be implemented as one or more processors in some embodiments.

The volume renderer 134 is configured to generate an image (also referred to as a projection, render, or rendering) of the 3D data set as viewed from a given reference point, as described for example by Entrekin et al. in U.S. Pat. No. 6,530,885, which is hereby incorporated by reference in its entirety. The volume renderer 134 may be implemented as one or more processors in some embodiments. The volume renderer 134 may generate a render, such as a positive render or a negative render, by any known or future known technique, such as surface rendering and maximum intensity rendering, for example.

In some embodiments, the system may include a Doppler signal path 152 which couples the output from the signal processor 126 to a Doppler processor 153. The Doppler processor 153 may be configured to estimate the Doppler shift and generate Doppler image data. The Doppler image data may include color data which is then overlaid with B-mode (i.e. grayscale) image data for display. The Doppler processor 153 may be configured to filter out unwanted signals (i.e., noise or clutter associated with non-moving tissue), for example using a wall filter. The Doppler processor 153 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some embodiments, the velocity and/or power estimates may undergo further threshold detection to further reduce noise, as well as segmentation and post-processing such as filtering and smoothing. The velocity and/or power estimates may then be mapped to a desired range of display colors in accordance with a color map. The color data, also referred to as Doppler image data, may then be coupled to the scan converter 131, where the Doppler image data may be converted to the desired image format and overlaid on the B-mode image of the tissue structure to form a color Doppler or a power Doppler image. In some examples, the scan converter 131 may align the Doppler image and B-mode image

Outputs from the scan converter 131, the multiplanar reformatter 132, and/or the volume renderer 134 are coupled to the image processor 136 for further enhancement, buffering and temporary storage before being displayed on an image display (e.g., display 113). The graphics processor 140 may generate graphic overlays for display with the images. These graphic overlays may contain standard identifying information, for example, such as patient name, date and time of the image, imaging parameters, and the like. For these purposes, the graphics processor 140 may be configured to receive input from the user interface 110, such as a typed patient name or other annotations. The user interface 110 may also be coupled to the multiplanar reformatter 132 for selection and control of a display of multiple multiplanar reformatted (MPR) images.

The image generation and processing components 130 further include local memory 142, which may be implemented as any suitable non-transitory computer readable medium, as discussed below with reference to memory 520 in FIG. 5. The local memory 142 may store executable instructions, training data sets, data generated by the ultrasound imaging system 100 including ultrasound images, and/or any other information necessary for the operation of the ultrasound imaging system 100. Although not all connections are shown to avoid obfuscation of FIG. 3, the local memory 142 may be accessible by additional components, in addition to the scan converter 131, the multiplanar reformatter 132, and the image processor 136. For example, the local memory 142 may be accessible to the graphics processor 140, the transmit controller 120, the signal processor 126, the user interface 110, and the like.

In some embodiments, various components shown in FIG. 3 may be combined. For instance, in some examples, a single processor may be configured to implement multiple components of the processing circuitry 150 (e.g., image processor 136, graphics processor 140) as well as the processing unit 115 in the user interface 110. In some embodiments, various components shown in FIG. 3 may be implemented as separate components. For example, signal processor 126 may be implemented as separate signal processors for each imaging mode (e.g., B-mode, Doppler, SWE). In some embodiments, one or more of the various processors shown in FIG. 3 may be implemented by general purpose processors and/or microprocessors, as discussed below with reference to memory 520 in FIG. 5, configured to perform the specified tasks. In some embodiments, one or more of the various processors may be implemented as application specific circuits. In some embodiments, one or more of the various processors (e.g., image processor 136) may be implemented with one or more graphical processing units (GPU).

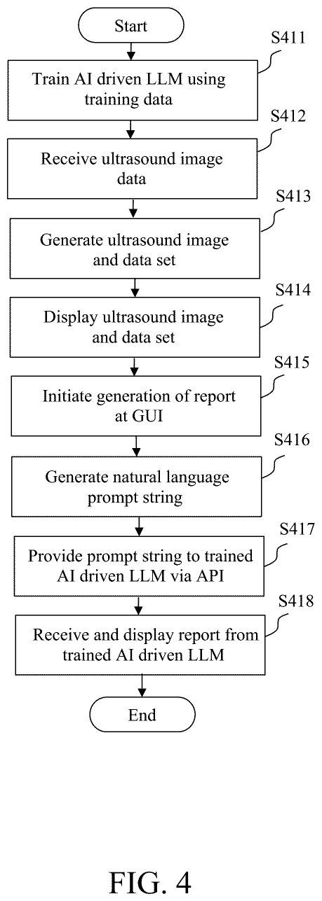

FIG. 4 is a flow diagram of a method for automatically generating a report from ultrasound imaging of a subject using a trained AI driven large language model, according to a representative embodiment. The steps in FIG. 4 may be implemented by the processing unit 115 and the AI server 101, for example, executing instructions stored in memory.

Referring to FIG. 4, the method includes training an AI driven large language model in block S411 using retrospective training data, as discussed above. The training may include receiving the retrospective training data from one or more databases collected from previous procedures. The retrospective data includes input training data and ground truth data corresponding to the input training data. The input training data includes natural language terms from training sets of patients subject to ultrasound imaging, types of ultrasound imaging devices, roles and experience of users performing the ultrasound imaging, and corresponding reports and report formats based on the ultrasound imaging including diagnoses. The input training data may further include information about the patients themselves, including patient information retrieved from EHRs, for example.

In block S412, the ultrasound image data acquired by an ultrasound imaging device is received by at least one processor, e.g., during an examination of the subject. The ultrasound image data includes echo data for generating an ultrasound image and corresponding metadata.

In block S413, an ultrasound image is generated by an ultrasound imaging device based on the ultrasound image data, and a data set is generated by the ultrasound imaging device indicative of measurements in the ultrasound image. The measurements may be made manually by the user setting calipers in the ultrasound image, or automatically by a trained machine learning auto-measurement model that automatically identifies and measures predetermined parameters in the ultrasound image when the correct anatomy is present.

In block S414, the ultrasound image and the data set are displayed on a display for the ultrasound imaging device. The display also provides a GUI that includes multiple configurable elements. Additional contextual information and data may be added via the GUI.

In block S415, a report generation is initiated by the user regarding the examination of the subject using the GUI provided on the display. To initiate the report, the user provides information (user input) in addition to the ultrasound image and data set for the report using one or more configurable elements of the GUI, and requests generation of the report by selecting a report selector (e.g., a button, a check box or a toggle) of the GUI. The additional information may include at least the role of to be simulated by the trained AI driven large language model, the role of the user, and the type of report desired. The additional information may also include patient data, which is specific information regarding the subject of the ultrasound imaging examination, such as age, weight, gender, vital signs, and/or all or part of the subject's medical history, and/or evaluation data, which is specific background information regarding the subject of the ultrasound imaging, such as information about the fetus, for example, as discussed above.

In block S416, a natural language prompt string is generated from at least the data set from the ultrasound imaging device and the user input from the GUI using a prompt string generator. The generation of the prompt string may be initiated in response to the selection of the report selector of the GUI, and may include translating the ultrasound image data, the data set and any user information into a predetermined template. The prompt string is suitable for the type of examination and is readable by the AI driven large language model.

In block S417, the prompt string is provided to the trained AI driven large language model using an API. That is, prompt string is output to an API, which calls the trained AI driven large language model to process the prompt string. The trained AI driven large language model automatically generates the report suitable for the type of examination and the user input. The report may include a diagnosis and a summary of the data set, for example, in a format or type of report indicated by the prompt string.

In block S418, the report is received from the trained AI driven large language model and displayed on the display. The report is reviewed by the user, who edits the contents using the GUI, if needed. After review, the report may be finalized.

FIG. 5 is a simplified block diagram of a representative processing unit, such as the AI server 101 and the processing unit 115 in FIG. 1, and one or more of the processors in the processing circuitry 150 of FIG. 3, according to a representative embodiment.

Referring to FIG. 5, processing unit 500 includes a processor 510, memory 520 for storing instructions executable by the processor 510 to implement the processes described herein, as well as a display 530 and an interface 540 to enable interactions with a user and/or other processing circuitry. The processor 510 is representative of one or more processing devices, and is configured to execute software instructions to perform functions as described in the various embodiments herein. The processor 510 may be implemented by a general purpose computer, a central processing unit, a graphical processing unit (GPU), one or more microprocessors or microcontrollers, a state machine, a programmable logic device, field programmable gate arrays (FPGAs), application specific integrated circuits (ASICs), or combinations thereof, using any combination of hardware, software, firmware, hard-wired logic circuits, or combinations thereof. The term “processor,” in particular, encompasses an electronic component able to execute a program or machine executable instructions. References to a processor should be interpreted to include more than one processor or processing core, as in a multi-core processor, and/or parallel processors. Programs have software instructions performed by one or multiple processors that may be within the same computing device or which may be distributed across multiple computing devices.

The memory 520 (as well as local memory 142) may include a main memory and/or a static memory, where such memories may communicate with each other and the processor 510 via one or more bus(es) 560. The memory 520 stores instructions used to implement some or all aspects of methods and processes described herein, including the methods described above with reference to FIG. 4, for example. The memory 520 may be implemented by any number, type and combination of random access memory (RAM) and read-only memory (ROM), for example, and may store various types of information, such as software algorithms, data based models including neural network based models, and computer programs, all of which are executable by the processor 510. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, blu-ray disk, a universal serial bus (USB) drive, or any other form of storage medium known in the art.

The memory 520 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term non-transitory specifically disavows fleeting characteristics such as characteristics of a carrier wave or signal or other forms that exist only transitorily in any place at any time. The memory 520 may store software instructions and/or computer readable code that enable performance of various functions. The memory 520 may be secure and/or encrypted, or unsecure and/or unencrypted.

“Memory” is an example of computer-readable storage media, and should be interpreted as possibly being multiple memories or databases. The memory or database may for instance be multiple memories or databases local to the computer, and/or distributed amongst multiple computer systems or computing devices. A computer readable storage medium is defined to be any medium that constitutes patentable subject matter under 35 U.S.C. § 101 and excludes any medium that does not constitute patentable subject matter under 35 U.S.C. § 101. Examples of such media include non-transitory media such as computer memory devices that store information in a format that is readable by a computer or data processing system. More specific examples of non-transitory media include computer disks and non-volatile memories.

Similarly, the database 550 may be implemented by any number, type and combination of RAM and ROM, for example, discussed above, The database 550 likewise is a tangible storage medium for storing data and executable software instructions, and is non-transitory during the time software instructions are stored therein. For example, the database may store retrospective data for training the AI driven large language model and/or patient data retrieved by the prompt string generator 116 to be included in the prompt string for the AI driven large language model, for example. The database 550 may be secure and/or encrypted, or unsecure and/or unencrypted.

The display 530 may be a monitor as described above with reference to display 113 in FIG. 1, and may provide a GUI for displaying and receiving information to and from the user. The interface 540 may include a user and/or network interface for providing information and data output by the processor 510 and/or the memory 520 to the user and/or for receiving information and data input by the user. That is, the interface 540 enables the user to enter data and to control or manipulate aspects of the processes described herein, and also enables the processor 510 to indicate the effects of the user's control or manipulation, as discussed above with reference to control panel 112 in FIG. 1. The interface 540 may also enable communication with other processing circuitry, including one or more other processing units and/or databases. The interface 540 may further include one or more of ports, disk drives, wireless antennas, or other types of receiver circuitry.

Although the present specification describes components and functions that may be implemented in particular embodiments with reference to particular standards and protocols, the disclosure is not limited to such standards and protocols. Such standards are periodically superseded by more efficient equivalents having essentially the same functions. Accordingly, replacement standards and protocols having the same or similar functions are considered equivalents thereof.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72 (b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A system for generating a report from ultrasound imaging, the system comprising:
an ultrasound imaging system (100) configured to provide ultrasound image data from an examination of a subject; and
a server (101) configured to execute one or more trained, artificial intelligence (AI) driven large language models configured to generate natural language responses to natural language input,
wherein the ultrasound imaging system comprises:
at least one processor (115, 510) configured to receive the ultrasound image data acquired by an ultrasound imaging device during the examination of the subject, including corresponding metadata, to generate an ultrasound image based on the ultrasound image data, and to generate a data set indicative of a plurality of measurements in the ultrasound image;
a display (113, 530) configured to display the ultrasound image and the data set;
a graphical user interface (GUI) (114) provided on the display, the GUI including a report selector selectable by a user to request a report regarding the examination of the subject and one or more configurable elements for receiving user input from the user; and
a prompt string generator (116) configured (i) to receive the user input from the GUI, (ii) to extract at least a type of examination and the data set from the at least one processor, (iii) to translate the user input, the type of examination, and the data set into a natural language prompt string in response to selection of the report selector, and (iv) to output the prompt string to an application programming interface (API) (117), wherein the prompt string is suitable for the type of examination and readable by the AI driven large language model,
wherein the AI driven large language model receives the prompt string via the API and automatically generates the report suitable for the type of examination and the user input, wherein the report includes a summary of the data set, and
wherein the display is further configured to receive and display the report from the AI driven large language model.

2. The system of claim 1, wherein the one or more configurable elements of the GUI include at least a first element for receiving indication of a role of the AI driven large language model, and a second element for receiving indication of a profession of the user,
wherein the AI driven large language model determines a type of report based at least in part on the role of the AI driven large language model and the profession of the user, and automatically generates the report according to the determined type of report.

3. The system of claim 2, wherein the one or more configurable elements of the GUI further include a third element for receiving indication of a target recipient of the report, including a language of the target recipient,
wherein the AI driven large language model determines the type of report further based on the target recipient.

4. The system of claim 1, wherein the one or more configurable elements of the GUI include a fourth element for receiving indication of a type of report,
wherein the AI driven large language model automatically generates the report according to the received type of report.

5. The system of claim 1, wherein the report is customized in a predetermined user format provided by the user input.

6. The system of claim 1, wherein the report button is a touchscreen button on the display.

7. The system of claim 1, wherein the one or more configurable elements of the GUI include a data set selection element for receiving a selection of the data set, and wherein the report button is enabled after the selection of the data set.

8. The system of claim 7, wherein the GUI is configured to display the selected data set on the display, and to enable the user to edit the selected data set prior to the selected data set being provided to the prompt string generator.

9. The system of claim 1, wherein the GUI is configured to display the prompt string on the display, and to enable the user to edit the prompt string prior to the prompt string generator outputting the prompt string to the API.

10. The system of claim 1, wherein the data set is formatted as a draft report, and the report suitable for the type of examination generated by the AI driven large language model is formatted as a final report.

11. The system of claim 1, wherein the server is integrated with the ultrasound imaging system.

12. The system of claim 1, wherein the server comprises a remote computer accessible by the ultrasound imaging system via a communications network.

13. The system of claim 1, each of the configurable elements comprises a text field, a button, or a drop down list.

14. A non-transitory computer readable medium storing instructions for generating a report from ultrasound imaging that, when executed by one or more one processors, cause the one or more processors to:
receive ultrasound image data acquired by an ultrasound imaging device during an examination of a subject, the ultrasound image data including corresponding metadata;
generate an ultrasound image based on the ultrasound image data;
generate a data set indicative of a plurality of measurements in the ultrasound image;
cause the ultrasound image and the data set to be displayed on a display;
receive a request for a report regarding the examination of the subject from a graphical user interface (GUI) in response to selection by a user of a report selector displayed by the GUI;
receive user input from the user in response to one or more configurable elements displayed by the GUI;
translate the user input, the data set and a type of examination into a natural language prompt string in response to the selection of the report selector, wherein the prompt string is suitable for the type of examination and readable by a trained, artificial intelligence (AI) driven large language model configured to generate natural language responses to the natural language prompt string;
output the prompt string to an application programming interface (API) configured to interface with the AI driven large language model;
automatically generate the report suitable for the type of examination and the user input using the AI driven large language model, wherein the report includes a summary of the data set; and
cause the report generated by the AI driven large language model to be displayed on the display.

15. The non-transitory computer readable medium of claim 14, wherein the one or more configurable elements of the GUI include at least a first element for receiving indication of a role of the AI driven large language model, and a second element for receiving indication of a profession of the user,
wherein the AI driven large language model determines a type of report based at least in part on the role of the AI driven large language model and the profession of the user, and
wherein the instructions cause the one or more processors to automatically generate the report according to the determined type of report.

16. The non-transitory computer readable medium of claim 14, wherein the one or more configurable elements of the GUI further include a third element for receiving indication of a target recipient of the report, including a language of the target recipient, and
wherein the AI driven large language model determines the type of report further based on the target recipient.

17. The non-transitory computer readable medium of claim 1, wherein the one or more configurable elements of the GUI include a fourth element for receiving indication of a type of report, and
wherein the instructions cause the one or more processors to automatically generate the report according to the received type of report.

18. A method of generating a report from ultrasound imaging, the method comprising:
receiving ultrasound image data acquired by an ultrasound imaging device during an examination of a subject (S412), the ultrasound image data including corresponding metadata;
generating an ultrasound image based on the ultrasound image data (S413);
generating a data set indicative of a plurality of measurements in the ultrasound image (S413);
displaying the ultrasound image and the data set on a display (S414);
receiving a request for a report regarding the examination of the subject from a graphical user interface (GUI) in response to selection by a user of a report selector displayed by the GUI (S415);
receiving user input from the user in response to one or more configurable elements displayed by the GUI (S415);
translating the user input, the data set and a type of examination into a natural language prompt string in response to the selection of the report selector (S416), wherein the prompt string is suitable for the type of examination and readable by a trained, artificial intelligence (AI) driven large language model configured to generate natural language responses to the natural language prompt string;
outputting the prompt string to an application programming interface (API) configured to interface with the AI driven large language model (S417);
generating the report suitable for the type of examination and the user input using the AI driven large language model, wherein the report includes a summary of the data set (S418); and
displaying the report generated by the AI driven large language model on the display (S418).

19. The method of claim 18, further comprising:
displaying the prompt string on the GUI; and
receiving at least one edit to the prompt string by the user via the GUI prior to outputting the prompt string to the API.

20. The method of claim 18, wherein the one or more configurable elements of the GUI include a data set selection element for receiving a selection of the data set, and wherein the report button is enabled after the selection of the data set.
